(19) United States
(12) Patent Application Publication
(10) Pub. No.: US 2005/0176809 A1
(43) Pub. Date: Aug. 11, 2005

(54) Method and compositions for treatment of painful disorders

(75) Inventor: Joel E. Bernstein, Deerfield, IL (US)

Correspondence Address: JONES DAY, 77 WEST WACKER, CHICAGO, IL, 60601-1692, US

(73) Assignee: Rodlen Laboratories, Inc., Vernon Hills, IL (US)

(21) Appl. No.: 10/772,809

(22) Filed: Feb. 5, 2004

(51) Int. Cl. A61K 31/551; A61K 31/5415

(52) U.S. Cl. 514/450; 514/220; 514/225.8; 514/629

(57) ABSTRACT

Chronic pain is treated with a combination of a standard dose of a non-narcotic analgesic and a low dose of a tricyclic antidepressant compound. The invention is effective in the treatment of chronic pain associated with neuropathic or fibromuscular disorders, where such pain has been unresponsive to non-narcotic analgesics alone.

This case is related to prior application Ser. No. 09/977,619. No benefit of priority of the prior case is claimed herein.

### BACKGROUND OF THE INVENTION

Pain is the most common and among the most troubling manifestations of a variety of diseases ranging from arthritis to cancer. A wide variety of analgesics have been employed to relieve or ameliorate pain. No single analgesic is uniformly effective and the use of many of these agents is limited by side effects or substance abuse profiles. Some painful disorders have been particularly resistant to treatment and these include chronic neuropathic pain syndromes such as postherpetic neuralgia and painful diabetic neuropathy, as well as other chronic painful disorders such as painful fibromuscular diseases.

Tricyclic antidepressants, usually prescribed for relief of mental depression, have also been less commonly administered for amelioration of chronic painful neuropathic and fibromuscular disorders. When administered orally for such pain relieving effects, the tricyclic compounds are provided in relatively large daily dosages of 100-200 mg/day, with “extreme” doses in the range of 25-300 mg/day. (Hardman J G, Limbird L E, Editors. Goodman & Gilman's The Pharmacological Bases of Therapeutics, Ninth Edition, 1996, McGraw Hill, New York, Page 433).

The non-narcotic analgesic acetaminophen and the nonsteroidal anti-inflammatory drugs (NSAIDs) are a heterogeneous group of chemical compounds which have proved very useful in treating many types of common acute pain, such as headache or backache, as well as the chronic pain associated with osteoarthritis. However, these compounds have been generally viewed as without clear beneficial effects on chronic neuropathic or fibromuscular pain and are not, therefore, widely utilized in the treatment of such disorders.

I have discovered, surprisingly, that the oral administration of low doses of tricyclic antidepressants concomitantly with the administration of a non-narcotic analgesic such as acetaminophen or an NSAID such as aspirin or ibuprofen produces unexpectedly dramatic amelioriation of pain in patients with chronic painful neuropathic or fibromuscular disorders. An additional benefit of the invention is that side effects commonly observed with the tricyclic antidepressants such as sedation and anticholinergic effects (e.g. dry mouth) are rarely observed when administered in such a combination.

The present invention relates to a method and compositions for treating chronic painful conditions such as chronic painful neuropathic pain and chronic painful fibromuscular disorders. The principal object of the present invention is to provide an oral remedy to patients suffering from chronic painful neuropathic or fibromuscular disorders in forms such as oral suspensions, tablets or capsules containing a low dose of a tricyclic antidepressant compound combined with a non-narcotic analgesic. The use of such a combination results in an unexpectedly dramatic reduction in pain symptoms for sufferers of such disorders. Additionally, side effects commonly observed with the tricyclic antidepressant component of such a combination product are greatly reduced.

This and other objects of the present invention may be more readily understood when considered in conjunction with the following detailed description and examples.

### DETAILED DESCRIPTION OF THE PREFERRED EMBODIMENTS

The present invention defines a “low dose” of a tricyclic antidepressant to be about 25 mg/day or less.

In a preferred embodiment of the invention, a patient experiencing chronic pain such as neuropathic or fibromuscular pain is treated with a combination of a standard dose of a non-narcotic analgesic and a low dose of a tricyclic antidepressant compound.

The non-narcotic analgesic is preferably selected from the group consisting of acetaminophen, and NSAID's. Commonly used NSAID's include aspirin, ibuprofen, flurbiprofen, ketoprofen, and naproxen. Standard doses of such non-narcotic analgesics can be in the range of about 0.50 grams to about 2.6 grams daily for a typical adult. The standard dosage can vary depending on factors such as the size and age of the patient, as is known in the medical arts. Standard doses of non-narcotic analgesics are typically in the range of about 0.50-2 gm/day for acetaminophen, about 0.6-2.6 gm/day for aspirin, and about 0.6-1.8 gm/day for ibuprofen.

The tricyclic antidepressant compounds used in the practice of the invention are preferably selected from the group consisting of doxepin, amitriptyline, desipramine, imipramine, and physiologically acceptable acid addition salts thereof. Other tricyclic antidepressant compounds and their physiologically acceptable acid addition salts also may find utility in the instant invention. Such physiological acid addition salts can be selected from the group consisting of hydrochloride, hydrobromide, hydroiodide, acetate, valerate, and oleate. The tricyclic anti-depressant compounds are administered in the range of about 2.5 mg to about 25 mg daily, preferable in the range of about 5 mg to about 20 mg daily, and more preferably about 10-15 mg daily.

The combination of non-narcotic analgesic and tricyclic antidepressant compound can be administered in the form of two separate preparations taken one right after the other. Alternatively, the combination can be present in a single composition in a pharmaceutically acceptable vehicle for oral administration. Such a composition and vehicle can be in a form selected from the group consisting of tablets, capsules, caplets, oral solutions, and oral suspensions.

I investigated the possible pain relieving effects of combinations of non-narcotic analgesics with low doses of tricyclic antidepressants by having patients with chronic pain ingest low doses of doxepin hydrochloride along with either acetaminophen, aspirin or ibuprofen. Patients ingesting such combinations noted not only surprisingly good relief of pain, but none of the troublesome side effects that usually accompany tricyclic antidepressant treatment of chronic pain.

The following examples further illustrate the invention.

### EXAMPLE 1

A 56 year old woman with widespread pain in the neck, back and arms associated with fibromyalgia unresponsive to aspirin or acetaminophen by themselves was administered doxepin 5 mg along with acetaminophen 500 mg before retiring for the night (i.e., at H.S.). The woman noted markedly reduced pain the next day and continued to take the combination of 5 mg doxepin and 500 mg acetaminophen for the next four months with excellent relief of myofascial pain.

### EXAMPLE 2

A 58 year old man with chronic osteoarthritic pain in the small joints of the extremities unresponsive to traditional doses of non-narcotic analgesics taken alone ingested a combination of 5 mg doxepin and 650 mg aspirin twice daily. The patient noted considerably less pain and stiffness in his joints and did not suffer from any drowsiness or dry mouth, side effects associated with larger doses of doxepin.

### EXAMPLE 3

A 60 year old woman with widespread pain in the neck, shoulders, arms and legs, unresponsive to oral NSAID therapy, accompanied by frequent headaches, non-restorative sleep and fatigue was orally administered doxepin 10 mg combined with ibuprofen 600 mg at H.S. While the patient's pain had previously been unresponsive to ibuprofen, the patient now received substantial pain relief including fewer headaches, as well as more restful sleep at night.

It will apparent to those skilled in the art that only some of the preferred embodiments have been described by way of example and that there are various modifications that fall within the scope of this invention.

1. A method for treatment of chronic pain comprising orally administering a combination of a low dose of a tricyclic antidepressant compound and a standard dose of a non-narcotic analgesic.

2. The method of claim 1 wherein said tricyclic antidepressant is administered in a dosage of from about 2.5 mg to about 25 mg daily.

3. The method of claim 1 wherein said tricyclic antidepressant compound is selected from the group consisting of doxepin, amitriptyline, desipramine, imipramine and physiologically acceptable acid addition salts thereof.

4. The method of claim 1 wherein said physiologically acceptable acid addition salts are selected from the group consisting of the hydrochloride, hydrobromide, hydroiodide, acetate, valerate and oleate.

5. The method of claim 1 wherein said non-narcotic analgesic is administered in a dosage from about 0.50 gms to about 2.6 gms daily.

6. The method of claim 1 wherein said non-narcotic analgesic is selected from the group consisting of acetaminophen and NSAIDs.

7. The method of claim 1 wherein said low dose of tricyclic antidepressant compound and said standard dose of non-narcotic analgesic are present in a single composition including a pharmaceutically acceptable vehicle for oral administration.

8. The method of claim 7 wherein said composition is in a form selected from the group consisting of tablets, capsules, caplets, oral solutions, and oral suspensions.

9. A composition for treatment of chronic pain comprising a combination of a low dose of a tricyclic antidepressant compound and a standard dose of a non-narcotic analgesic in a pharmaceutical acceptable vehicle for oral administration.

10. The composition of claim 9 wherein said tricyclic antidepressant compound is administered in a dosage of from about 2.5 mg to about 25 mg daily.

11. The composition of claim 9 wherein said tricyclic antidepressant compound is selected from the group consisting of doxepin, amitriptyline, desipramine, imipramine, and physiologically acceptable acid addition salts thereof.

12. The composition of claim 9 wherein said physiologically acceptable acid addition salts are selected from the group consisting of the hydrochloride, hydrobromide, hydroiodide, acetate, valerate and oleate.

13. The composition of claim 9 wherein said non-narcotic analgesic is administered in a dosage for from about 0.50 gms to about 2.6 gms daily.

14. The composition of claim 9 wherein said non-narcotic analgesic is selected from the group consisting of acetaminophen or NSAIDs.

15. The composition of claim 7 wherein the combination of a tricyclic antidepressant and a non-narcotic analgesic and a pharmaceutically acceptable vehicle is in a form selected from the group consisting of tablets, capsules, caplets, oral solutions and oral suspensions.
